(19) United States
(12) Plant Patent Application Publication
(10) Pub. No.: US 2003/0192100 P1
(43) Pub. Date: Oct. 9, 2003

(54) Chrysanthemum plant named 'Chamanda'

(76) Inventor: Jean-Pierre Challet, Nuaille (FR)

Correspondence Address: BURNS, DOANE, SWECKER & MATHIS, L.L.P., P.O. Box 1404, Alexandria, VA, 22313-1404, US

(21) Appl. No.: 10/114,954

(22) Filed: Apr. 4, 2002

(51) Int. Cl. A01H 5/00

(52) U.S. Cl. PLT/291

(57) ABSTRACT

A new and distinct Chrysanthemum cultivar is provided that is the result of a controlled breeding program. Attractive bicolored blossoms having light rose ray florets and cherry red disc florets are formed in profusion. Such blossoms commonly are approximately 7 cm in diameter when fully open. The growth habit is low and compact and the plant branches well following pinching. The foliage is dark green and contrasts well with the bicolored blossom coloration. The plant grows well in pots and displays a very regular culture character.

### BOTANICAL/COMMERCIAL CLASSIFICATION

Dendranthema grandifloria/Decorative Pot Mum

### VARIETAL DENOMINATION

cv. ‘Chamanda’

### SUMMARY OF THE INVENTION

The present invention comprises a new and distinct cultivar of Chrysanthemum, botanically known as Dendranthema grandiflora, and hereafter is referred to by the cultivar name ‘Chamanda’.

The new cultivar of the present invention was created at Nuaillé, France during the course of a controlled breeding program. The seeds resulting from the cross were sown and small plants were obtained which were physically and biologically different from each other. Selective study resulted in the identification of a single plant of the new cultivar of the present invention.

It was found that the new Chrysanthemum cultivar of the present invention displays:

(a) a low compact growth habit with good branching following pinching,

(b) attractive long-lasting decorative bicolored blossoms having light rose ray florets and cherry red disc florets,

(c) attractive dark green leaves that contrast nicely with the bicolored blossom coloration, and

(d) an ability to grow well in pots.

The new cultivar can be grown singly or in clumps in pots. It also can be grown in the landscape. The bicolored blossoms contract nicely with the dark green foliage as illustrated. The blossoms have been found to be long lasting and commonly last approximately four weeks on the plant. Pinching is helpful to enhance branching.

The new cultivar displays very good regularity in culture.

Asexual reproduction of the new cultivar by the use of cuttings as performed at Nuaillé, France, in a controlled environment has demonstrated that the characteristics of the new cultivar are firmly fixed and are retained through successive generations of asexual propagation.

‘Chamanda’ has not been observed under all possible environmental conditions to date. Accordingly, it is possible that the phenotype may vary somewhat with variations in the environment, such as temperature, light, day length, contact with pesticides and/or subjection to grown regulation treatments.

### BRIEF DESCRIPTION OF THE PHOTOGRAPH

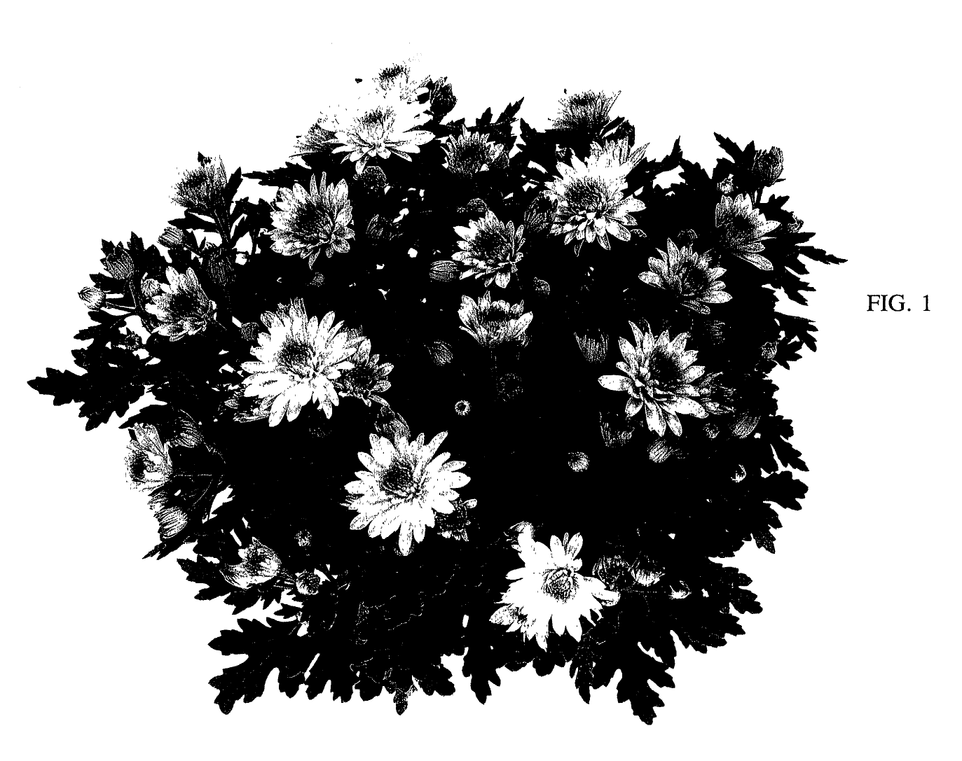

The accompanying FIG. 1 depicts the typical bicolored blossoms and foliage of the new ‘Chamanda’ cultivar while growing in a pot in a greenhouse at Nuaillé, France. The attractive blossoms having light rose ray florets and cherry red disc florets are illustrated. The illustrated plant had undergone pinching.

### DETAILED DESCRIPTION

The plants described were grown in pots in a greenhouse at Nuaillé, France. No growth regulation was used. The growing conditions approximate those commonly utilized for the commercial production of decorative pot mums.

General Appearance:

Short and stocky. Good branching is exhibited following pinching.

Foliage:

Attractive dark green (as illustrated).

Flowers:

Possess a decorative bicolored configuration. The ray florets are light rose in coloration and the disc florets are cherry red in coloration. There is some lightening of the coloration of the ray florets as the blossoms mature. The disc florets sometimes assume a purple coloration with full maturity. The diameter generally is approximately 7 cm when fully expanded.

Flower Duration:

Very long lasting with the blossoms commonly lasting approximately four weeks on the plant.

Culture:

Very regular.

Usage:

Decorative pot mum.

I claim:

1. A new and distinct cultivar of Chrysanthemum plant that displays:
(a) a low compact growth habit with good branching following pinching,
(b) attractive long-lasting decorative blossoms having light rose ray florets and cherry red disc florets,
(c) attractive dark green leaves that contrast nicely with the bicolored blossom coloration, and
(d) an ability to grow well in pots;
substantially as illustrated and described.
